(19) United States
(12) Patent Application Publication
(10) Pub. No.: US 2009/0218413 A1
(43) Pub. Date: Sep. 3, 2009

(54) Dispensing Devices

(75) Inventor: Philip Craig Withers, South Bank (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: INTELLECTUAL PROPERTY DEVELOPMENT CORPORATION PTY, Melbourne (AU)

(21) Appl. No.: 12/084,161

(22) PCT Filed: Oct. 24, 2006
(86) PCT No.: PCT/AU2006/001578

§ 371 Date: Feb. 19, 2009

(30) Foreign Application Priority Data
Oct. 24, 2005 (AU) 2005905887

(51) Int. Cl. A61L 9/04; A61L 9/12

(52) U.S. Cl. 239/6; 239/34

(57) ABSTRACT

A dispenser for dispensing at least first and second compositions stored in the dispenser, the dispenser comprising an adjustable discharging mechanism for discharging the first composition at a first rate and the second composition at a second rate. The discharging mechanism may include a first adjustable discharge mechanism for discharging the first composition at the first rate and a second adjustable discharge mechanism for discharging the second composition at the second rate.

### RELATED APPLICATION

This application is based on and claims the benefit of the filing date of Australian patent application no. 2005905887 filed 24 Oct. 2005, the content of which is incorporated herein by reference in its entirety.

### FIELD OF THE INVENTION

The present invention relates to dispensing devices for dispensing chemical materials into the atmosphere by evaporation or movement of air with respect to the chemical materials, particularly over the surface of the chemical compositions.

### BACKGROUND OF THE INVENTION

Various different types of air freshening devices for dispensing a variety of different air freshening compositions or similar compositions are available. One type of air care dispenser relies upon evaporation to dispense the contained air care composition, including the active component or components of the composition. Some of the currently available evaporation type air care containers have a compartment that is open to the atmosphere; the composition in the container is exposed to the atmosphere so that movement of air over the surface of the composition dispenses the composition from the container at a set or constant rate according to the rate of movement of the air. Such devices are not adjustable, so the rate at which the composition is dispensed is determined solely by the rate of air movement with respect to the device.

Other air care containers are available with some type of adjustment for selectively adjusting the rate of evaporation of the formulation contained within the device so that the rate at which the active components are dispensed can be varied. However, this is the only adjustability provided by such air care devices.

### SUMMARY OF THE INVENTION

According to a first broad aspect of the present invention there is provided a dispenser for dispensing at least first and second compositions stored in the dispenser, the dispenser comprising an adjustable discharging mechanism for discharging the first composition at a first rate and the second composition at a second rate.

The dispenser may include a first compartment for containing the first composition and a second compartment for containing the second composition. As will be appreciated, however, the dispenser will typically—but not necessarily—have as many compartments as it has compositions, whether two compositions or more. For example, four compositions might be provided in four compartments but, in other embodiments, two compositions might be provided in four compartments. Alternatively, two or more compositions could share a single compartment, so the number of compositions could in fact exceed the number of compartments.

In one embodiment, the discharging mechanism is adapted to permit the first rate and the second rate to be adjusted relative to each other.

Thus, the dispenser is controllable to discharge selected amounts of each of the first and second (or more) compositions such that the amount of material being dispensed has a selected composition or is dispensed in selected quantities, concentrations or rates of the compositions.

In one particular embodiment, the discharging mechanism includes a first adjustable discharge mechanism for discharging the first composition at a first rate and a second adjustable discharge mechanism for discharging the second composition at a second rate, wherein the first rate and the second rate are at least partially independently adjustable.

In one embodiment, the first composition is a first form of a composition and the second composition is a second form of the composition. The first composition (of this and other aspects of the invention) may be either the same as or different from the second composition.

The first rate may be either the same as or different from the second rate. It should also be noted that the independent adjustability of the first and second rates may allow either or both of the first and second rates to be reduced to zero.

In another embodiment, the discharging mechanism is adapted to expose the first composition and the second composition to different extents as the discharging mechanism is adjusted. For example, the discharging mechanism may comprise a rotatable shutter that in a first position fully covers the first composition, in a second position fully covers the second composition and in a third position covers the first composition partially and the second composition to a greater or lesser extent than the discharging mechanism covers the first composition. Alternatively, the discharging mechanism may comprise a closure for covering a vent from which the first and second compositions are discharged, wherein the closure has a plurality of positions that permit discharging of the first and second compositions at different rates; in one embodiment this is effected by providing a vent that permits more venting by the first composition than by the second composition.

Typically the first composition can be of any suitable form or type. More typically the compositions include air care products or compositions, such as for example, air care or air treatment compositions including odour neutralises, fragrances (such as lavender), perfumes, masking agents or the like. Typically the composition in the first compartment can be the same or different to the composition in the second compartment. More typically, the first and second compositions can be different concentrations of the same composition or of different compositions. More typically, the composition contains at least one substance that reacts with the olfactory senses, such as smell.

Typically the dispenser contains two or more compartments, typically, two, three, four or more separate compartments. More typically, at least two of the compartments share a common dividing wall. Typically, the compartments are fixed with respect to each other. Even more typically, the two compartments are movable relative to one another, typically about a common axis interconnecting the two parts of the dispenser.

Typically the discharging mechanism comprises one or more openings, spaces, gaps, discontinuities or the like allowing movement of air therethrough. Even more typically, the opening is a vent, aperture, port, window, louvre, bore or similar through which the composition is discharged. More typically, there are a multitude of openings or vents, typically arranged in groups of openings or vents, including spaced apart individual openings arranged with respect to each other. More typically, the opening or vent allows movement of air over the surface of the composition. More typically, the opening or vent is adjustable. More typically, the opening or vent is adjustable by being selectively movable between selected ones of multi positions or the vent is associated with a closure that is movable between selected ones of a multitude of positions. Typically, the openings can be selectively adjustable by having a moving adjustor. More typically, the adjustor is a depressible, slidable, rotatable, pivotable, twistable adjustor or the like. Typically the positions range from a fully closed position to a fully opened position. More typically there are multiple positions intermediate the extreme positions, typically from 2 to 10 or more adjustable positions, between fully closed and fully opened for controlling discharge of materials. Typically the discharging mechanism can adopt one of four or five or more separate positions; one being fully closed, one being fully opened, and two or three being intermediate positions that are partially opened positions. More typically, selected ones of the individual groups of vents are adjustably openable or the like.

Typically, the composition can be in the form of a viscous liquid, a gel, a solid or a combination of two or more. More typically, the compositions are homogeneous or a mixture. More typically the compositions are layered, laminated, or of a complex structure such as a multi-layered glove, sphere or other three dimensional solid shape or of a simple shape or structure or the like.

Typically the composition in the first compartment evaporates at a first rate which is determined by the position of the first discharge mechanism, that is, the extent of the opening allowing air to circulate or flow through the first compartment. Typically the second composition in the second compartment is discharged at a second rate determined by the position of the second discharge mechanism, being the opening of the second compartment. Typically, there are a multitude of openings and a multitude of flow paths for the air to travel through and around the dispenser.

However, the first and second rates may be relatively adjusted by adjusting the discharging mechanism to expose the first composition to a greater extent than the second composition.

The compartments may be of the same size or of a different size. More typically, the concentrations of compositions in the different compartments are in accordance with the size of the compartments either directly or indirectly. The compositions may be in the form of refills, inserts, replaceable cartridges, cassettes, packages or the like in which the individual containers can be replenished easily with a custom made replacement that fits easily in the container when it is time to renew the air freshener so that the container can be reused without having to purchase new containers.

Typically, the composition includes oils such as artificial fragrances, essential oils, aromatherapy oils or the like. Typically the composition is in the form of a liquid or a gel, typically a slow release gel, more typically a gel which is dispensed by the movement of air over the surface of the gel composition, more typically a variable release gel or the like. More typically, the gel is a volatile gel or is a slow release gel, a controlled release gel or the like or a similar form of a composition.

Typically any material that has a volatility can be included in the compositions, preferably a material having volatility at room temperature, preferably having an effective volatility at room temperature sufficient to discharge olfactory effective or responsive amounts of chemicals to atmosphere.

In one embodiment, the dispenser includes a removable seal for preserving the first and second compositions prior to use. The seal may be partially removable so control the discharge of one or both of the compositions.

The dispenser may include a first removable seal for the first composition and a second removable seal for the second composition, wherein the first removable seal can be partially or fully removed to partially or fully expose the first composition and the second removable seal can be partially or fully removed to partially or fully expose the second composition. The first removable seal may comprises a plurality of seal segments that can be individually removed to expose the first composition by respective different amounts.

According to a second broad aspect of the present invention there is provided a method of dispensing first and second compositions from a dispenser, comprising selectively adjusting a discharging mechanism to dispense the first composition at a first rate and the second composition at a second rate.

The dispenser used according to this method may have any of the characteristics of the above-described dispenser in the first aspect.

The discharging mechanism may comprise a first discharge mechanism for controlling discharge of the first composition at the first rate and a second discharge mechanism for controlling discharge of the second composition at the second rate, the method comprising independently adjusting the first discharge mechanism and the second discharge mechanism.

The first and second rates may be relatively adjusted to select a desired effect (of concentration, etc).

In one embodiment, the method includes discharging the first composition from a first compartment of the dispenser, and discharging the second composition from a second compartment of the dispenser.

The method may include independently controlling the first rate from a minimum (and possibly zero) rate to a maximum rate and the second rate from a minimum (and possibly zero) rate to a maximum rate. More typically, the minimum rate corresponds to the compartment being substantially fully closed and the maximum rate corresponds to the compartment being fully opened. More typically, the dispenser can adopt a transport configuration in which the dispenser is fully closed, and can adopt one of many in use configurations in which the dispenser is in one of the open conditions ranging from a partially opened configuration to a fully opened configuration. Typically, there is a multitude of partially opened configurations.

The method may include dispensing the compositions singly, in combination, simultaneously or sequentially.

The method may include combining, blending or mixing the first composition and the second composition as desired so that a selected blend of the first and the second compositions are dispensed from the dispenser, such as according to personal preferences or as desired so that a selected blend of the first and second compositions is dispensed from the dispenser. Typically, the selected blend of composition is selected for serving one or more of a number of different purposes. More typically, more of one composition, such as a fragrance, can be dispensed from one compartment or more of the other composition, such as a odour neutraliser, can be dispensed from the other compartment. More typically, any combination from 0% to 100% of one composition and from 100% to 0% of another composition can be dispensed from the dispenser either simultaneously, separately or sequentially.

Typically the dispenser could be heated to increase the discharge rate of the compositions. More typically, each compartment is provided with its own heater. Even more typically, the rate of discharge of any one composition is controlled by the opening of the vent and the amount of heating. More typically, each heater is independently controllable to control the rate of discharge of the individual compositions. Typically, the heat can be derived from solar panels or the like. More typically, the discharging mechanism can be controlled electronically, typically, computer controlled, such as for example by being networked to other appliances or the like, including air conditioners, or other devices.

The method may include dispensing one, each or both compositions at a rate of up to 100 g or 100 mL per day, preferably from about 0.01 g per day up to about 50 g or mL per day, preferably from about 0.1 up to about 10 g or mL per day, more preferably from 0.2 to 5 g or mL per day and even more preferably about 3 to about 4 g or mL per day, depending upon the form of the composition, such as for example whether it is liquid, solid, a gel, or the like with gels producing greater discharging rates than liquids or solids.

According to a third broad aspect of the present invention there is provided a refill for a dispenser for dispensing at least first and second compositions, comprising:
a container;
a first composition and a second composition; and
a removable seal for preserving said first and second compositions prior to use;
wherein said removable seal is partially removable, fully removable, or both partially and fully removable.

The container may have a first compartment for the first composition and a second compartment for the second composition.

The container may be adapted to fit a holder having an adjustable discharging mechanism for discharging the first composition at a first rate and the second composition at a second rate. The discharging mechanism may permit the first rate and the second rate to be adjusted relative to each other.

In one particular embodiment, the adjustable discharging mechanism includes a first adjustable discharge mechanism for discharging the first composition at a first rate and a second adjustable discharge mechanism for discharging the second composition at a second rate, wherein the first rate and the second rate are at least partially independently adjustable.

### BRIEF DESCRIPTION OF THE DRAWING

In order that the present invention may be more clearly ascertained, the invention will now be described by way of non-limiting examples with reference to the accompanying drawings in which:

FIG. 1 is a top perspective view of an air care dispenser according to an embodiment of the present invention, in a fully closed configuration;

FIG. 2 is a top perspective view of the lid of one compartment of the air care dispenser of FIG. 1;

FIG. 3 is a top perspective view of the base of the air care dispenser of FIG. 1;

FIG. 4 is an exploded view of the air care dispenser of FIG. 1 showing the relationship of the two lids to each other and to the base of the air care dispenser;

FIG. 5 is a top perspective view of the air care dispenser of FIG. 1 showing one lid in place and one lid removed shown in phantom and also showing the direction of air flow or air movement by the use of arrows;

FIG. 6 is a top perspective view of an air care dispenser according to another embodiment of the present invention, in a fully closed configuration;

FIG. 7 is a top perspective view of the air care dispenser of FIG. 6 in one of its in-use configuration;

FIG. 8 is an exploded view showing the assembly of the two movable compartments of the air care dispenser of FIG. 6 to each other;

FIG. 9 is a cross-sectional view of the air care dispenser of FIG. 6 when in the fully closed configuration;

FIG. 10 is a side elevation view of a dispenser according to still another embodiment of the present invention in an open configuration;

FIG. 11 is a longitudinal cross-section taken along the line II to II of FIG. 10;

FIG. 12 is an exploded perspective view of the dispenser of FIG. 10 showing the individual components forming the dispenser;

FIG. 13 is a top perspective view of an air care dispenser according to a further embodiment of the present invention, in a fully closed configuration;

FIG. 14 is a top perspective view of the air care dispenser of FIG. 13, with the cap of the dispenser removed from the base of the dispenser;

FIG. 15 is an exploded view of the air care dispenser of FIG. 13;

FIG. 16 is a top perspective view of the air care dispenser of FIG. 13, in an opened configuration;

FIG. 17 is a cross-sectional view of the air care dispenser of FIG. 13, in an opened configuration;

FIG. 18 is a detail of a cross-sectional view of the air care dispenser of FIG. 13, in an opened configuration;

FIG. 19 is another detail of a cross-sectional view of the air care dispenser of FIG. 13, in an opened configuration;

FIG. 20 is a partial bottom perspective view of the upper rim of the cap of the air care dispenser of FIG. 13;

FIG. 21 is a front perspective view of an air care dispenser according to a further embodiment of the present invention, in a closed configuration;

FIG. 22 is a rear perspective view of the air care dispenser of FIG. 21;

FIG. 23 is a partially exploded rear perspective view of the air care dispenser of FIG. 21;

FIG. 24 is an exploded rear perspective view of the front cover of the air care dispenser of FIG. 21;

FIG. 25 is a cross-sectional view of the air care dispenser of FIG. 21;

FIG. 26 is a front perspective view of the air care composition container of the air care dispenser of FIG. 21;

FIG. 27A is a top perspective view of a refill according to a further embodiment of the present invention, suitable for use with the dispenser of FIG. 13;

FIG. 27B is a top perspective view of the refill of FIG. 27A, shown in a partially open configuration;

FIG. 28 is a top perspective view of a refill according to another embodiment of the present invention, suitable for use with the dispenser of FIG. 13;

FIG. 29 is a top perspective view of a dispenser or refill according to a still further embodiment of the present invention, suitable for use with use with the dispenser of FIG. 13; and

FIG. 30 is a top perspective view of a dispenser or refill according to another embodiment of the present invention, suitable for use with the dispenser of FIG. 13.

### DETAILED DESCRIPTION OF THE EMBODIMENTS

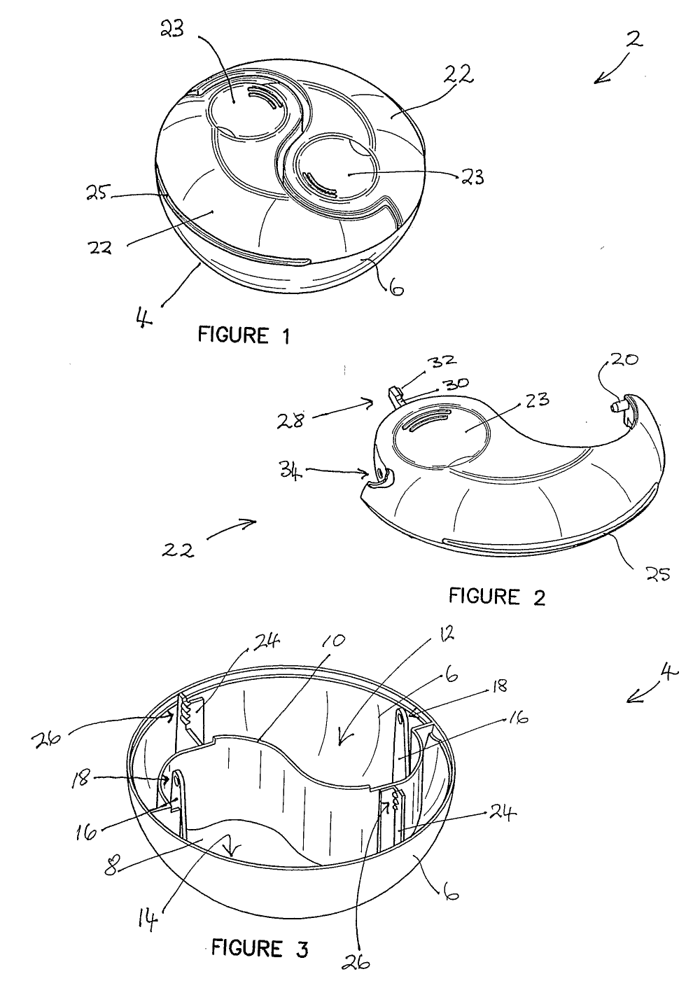

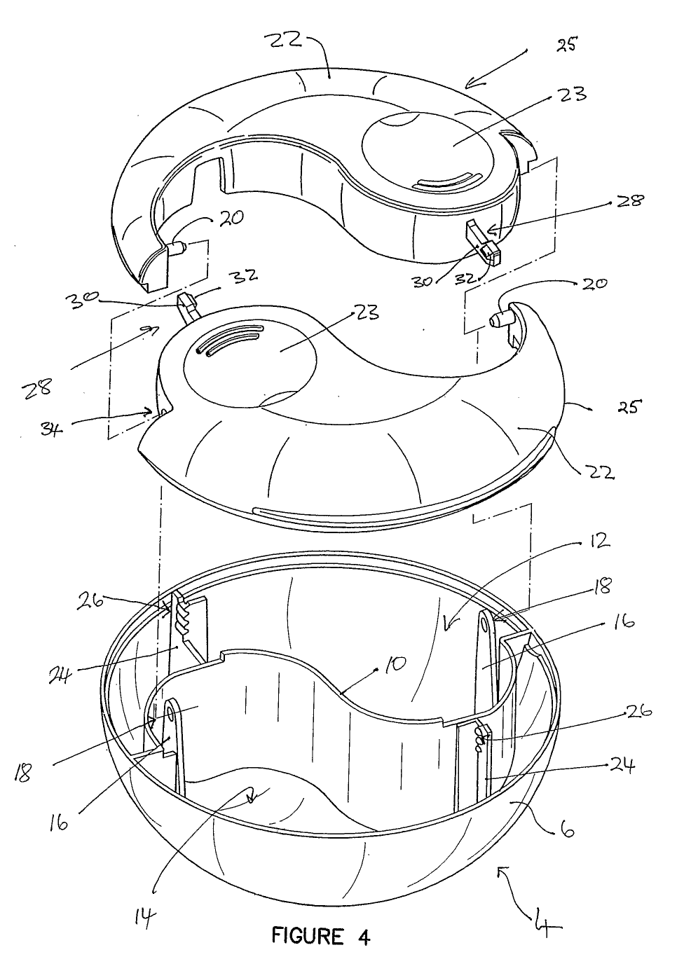

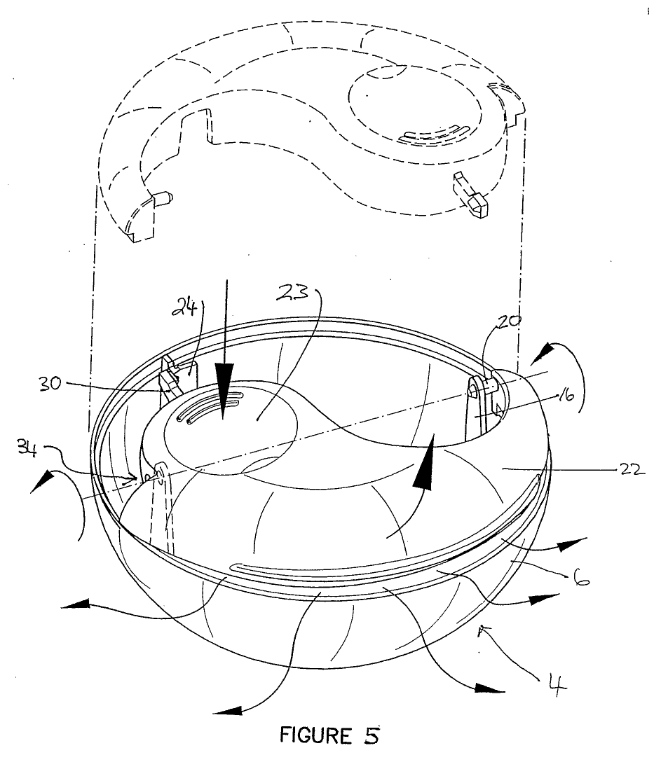

FIGS. 1 to 5 are views of an air care dispenser 2 according to a first embodiment of the present invention. The dispenser 2 has a base 4 and a discharging mechanism in the form of two rotatably mounted, tiltable lids 22; dispenser 2—apart from its air care composition contents—is of a plastics material (in common with the other embodiments described below). Base 4 takes the form of a generally round open top bowl having a circular cross section with arcuate side wall 6 curving to form a bottom surface having a flattened portion 8 upon which the dispenser 2 can rest or be supported upon a suitable solid substrate such as a shelf, bench or other substantially horizontal surface. The base 4 is divided into two separate compartments 12, 14 by substantially sinusoidal central wall 10 extending from one side of the base 4 to a diametrically opposite side in a curved or convolute manner. Although wall 10 is shown as being curved it is to be noted that in other embodiments of the container the wall can adopt different configurations such as for example a straight wall extending from one side of the base to the other along a diameter.

First compartment 12 is defined by a first portion of arcuate side wall 6 and central wall 10. Second compartment 14 is defined by a second portion of arcuate side wall 6 and central wall 10. A first composition (not shown) can be received and stored in first compartment 12 formed on one side of wall 10 and a second composition (not shown) can be received and stored in second compartment 14 formed on the other side of wall 10. In one form, both the first and second compositions are gels. However, the compositions can take or be provided in any suitable form including refills, inserts, replaceable packs or the like.

It is to be noted that both the first and second compartments 12, 14 are—in this embodiment—essentially identical so, for ease of description and clarity of understanding, as far as it is possible only first compartment 12 will be fully described. However, it is to be noted that the two compartments 12, 14 may—in other embodiments—be of different sizes or asymmetrical, or otherwise differ.

Thus, first compartment 12 is provided with a generally upstanding post 16 located at or towards one end of central wall 10. A circular aperture 18 is provided at and towards the top of post 16 for receiving spigot 20 of one of lids 22 (forming the top of the dispenser 2 and described in more detail below). Lid 22 is provided with depression 23 for assisting movement of the lid. Rib 24 is provided adjacent to central wall 10 towards the other end of wall 10 from post 16. Rib 24 can adopt any suitable form. In one form rib 24 is substantially L-shaped in transverse cross section and is provided with a multitude of vertically spaced apart notches 26 located co-linearly with respect to each other or towards the upstanding end of rib 24. In one embodiment there are four notches as shown in the drawings. However, in other embodiments there may be a different number of notches including fewer notches or more notches depending upon the degree of control and/or adjustability required for dispensing the composition in the compartment. The notches act as detents for complementary features of the lid to allow the lid to be selectively opened with respect to base 4 as will be described in more detail later.

As mentioned above, lids 22 constitute a discharging mechanism for controlling the discharge or dispensing of the first and second compositions; each lid 22 constitutes an independently adjustable discharge mechanism. Each lid 22 is of a generally curved shape having an outer surface 25 corresponding to the radius of curvature of the side wall 6 of base 4 and extending around half of the circumference of wall 6. Each lid 22 is provided with spigot 20 that is journalled in aperture 18 of post 16 to allow the respective lid 22 to rotate about a centre line or diameter joining one side of base 4 to a diametrically opposite side of base 4 as illustrated between selectively opened positions ranging between the fully closed and fully opened positions.

The inner side wall of each lid 22 is provided with arm 28 having an enlarged head 30 located at the distal end of arm 28. Enlarged head 30 is provided with a generally tapered projection 32 for co-operatively being received in a selected one of the notches 26. In use, when tapered projection 32 is received in the lowermost notch of rib 24, lid 22 is in a fully closed position whereas when tapered projection 32 is received in the uppermost notch, the lid 22 is in the fully opened position.

An aperture 34 is provided on the side of each lid 22 opposite spigot 20, so that the spigot 20 of one lid 22 is received through aperture 34 of the other lid 22 and through aperture 18 of post 16 to secure the lids 22 to base 4, as posts 16 are located diametrically opposite each other with respect to base 4.

In operation of dispenser 2, a first composition having a first evaporation rate and having a first selected fragrance or similar is introduced into first compartment 12. A second composition having a second fragrance is introduced into second compartment 14 so that there are two different compositions in the dispenser, one in each compartment. The two lids 22 are assembled into place on base 4 to form dispenser 2. When the lids 22 are in the fully closed condition as shown in FIG. 1 the dispenser 2 is in a transport condition, which prevents movement of air from within the dispenser to outside the dispenser so as to prevent evaporation of the two compositions and thus prevents dispensing of the compositions from the air care device. In use, the dispenser 2 is located in the desired location resting upon flat bottom 8. The first lid 22 is adjusted by selectively moving the flip lid 22 to move arm 30 so that the tapered projection 32 moves into position within the desired notch 26 to allow the lid 22 to adopt a selectively opened position to dispense the composition at the desired rate. It is to be noted that by pushing down on depression 23 of lid 22 on one side of the centre line between posts 16 raises the opposite side of lid 22 on the other side of the centre line so as to partially open the dispenser. In this position the composition is able to be dispensed from the respective compartment, as the opening allows movement of air through this side of the dispenser 2. As there are two lids, each lid 22 can be opened by pushing down on its respective depression 23 to a different extent so that the other side of the respective lid is opened by a different amount to dispense the contents of the two compartments at different rates from each other. For example, if an odour is to be overcome, first compartment 12 can contain an odour neutraliser and second compartment 14 can contain a fragrance; the odour neutraliser containing compartment 12 could be fully opened and the fragrance containing compartment 14 fully or partially closed. However if it is desired not to release any odour neutraliser, compartment 12 could be fully closed and the fragrance containing compartment 14 partially or fully opened to the desired amount. In other embodiments there can be different types of compositions opened to respective amounts by selectively opening lids 22 to allow greater or lesser amounts of air flow through each compartment 12, 14. Any combination of levels of opening can be selected to adjust the rate of dispensing required to dispense the selected amount of each composition. It is to be noted that the tapered projection 32 of the lid 22 of one compartment on one side of device 2 is located in the selected notch 26 provided within the compartment on the other side of device 2, and vice versa, so that both lids 22 interact with the other compartment over which it is located to adopted the selected position.

Thus, air care dispenser 2 of this embodiment of the present invention allows two different compositions to be dispensed at two different rates by independently selectively adjusting the position of the lids 22 by depressing the lids to different extents with respect to the base 4 by having the respective tapered projections 32 of the respective lids received in the respective notches 26 of the respective other compartment.

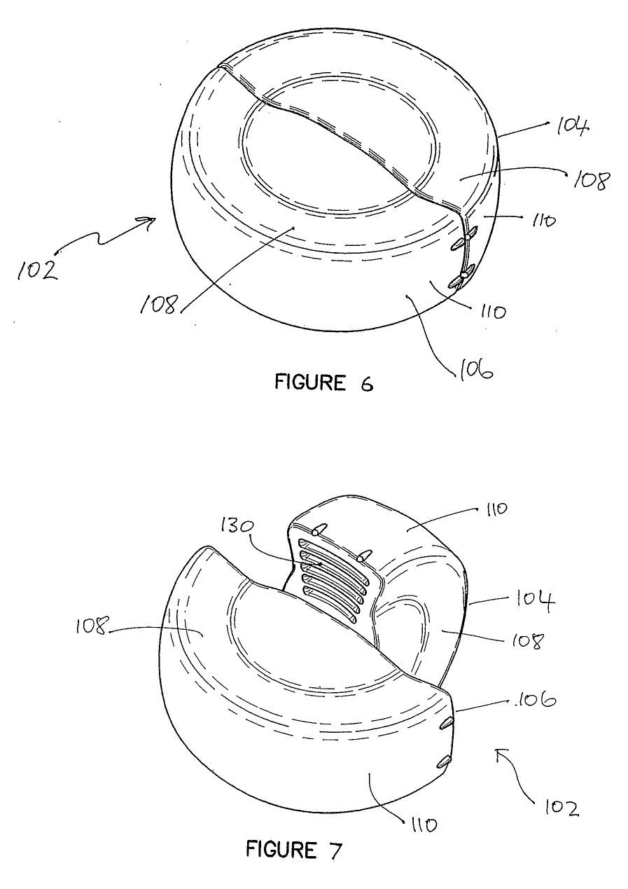

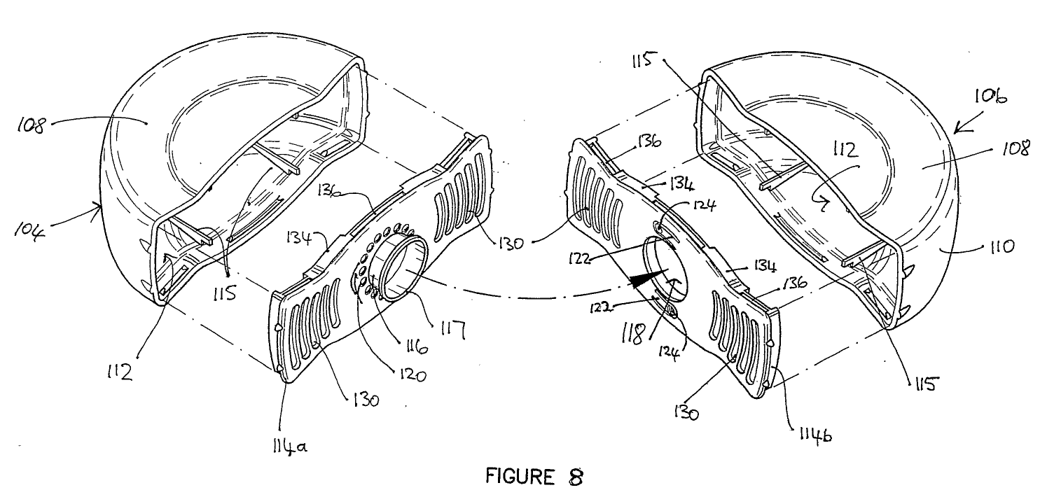

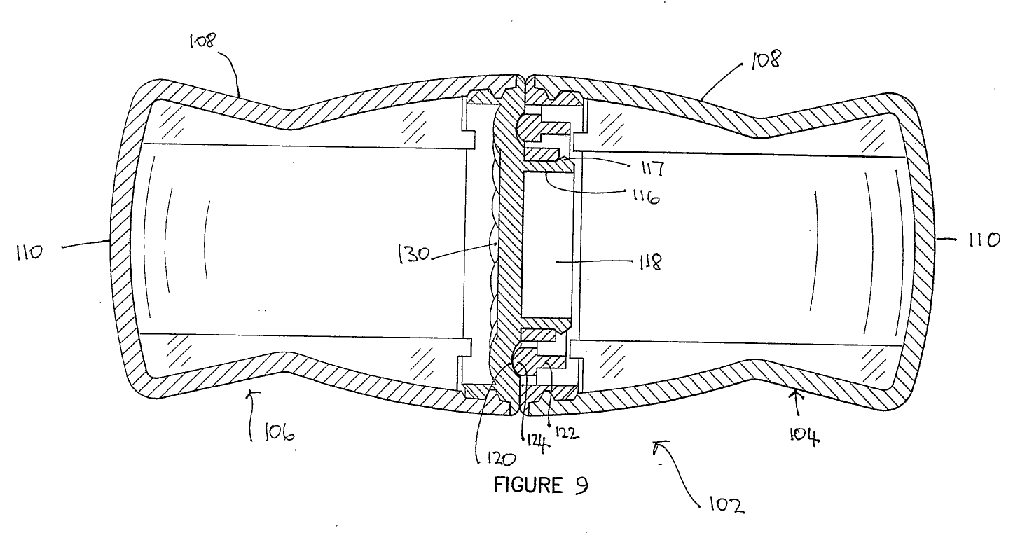

An air care dispenser 102 according to another embodiment of the present invention is shown in FIGS. 6 to 9. Dispenser 102 is substantially cylindrical, being formed from two semi-cylindrical compartments 104 and 106 interconnected to each other along an axis transverse to a central axis extending between them and forming a diameter wall of the centre line of the cylinder formed from the two semi-cylindrical compartments 104, 106.

Each half of this dispenser 102, that is, compartment 104 or 106, is a semi-circular hollow body 108 having a generally arcuate side wall 110 and an (otherwise) open central aperture 112 extending along the diameter wall subdividing the two compartments 104, 106. Each central aperture 112 is provided with a respective grille 114a, 114b, located in the respective central aperture 112. Hence, each compartment 104, 106 has its own respective grille 114a, 114b. A suitable fitting, such as a snap lock fitting in the form of a pair of outwardly extending ribs 115, is provided to enable respective grille 114a, 114b to be lockingly received in its corresponding central aperture 112. The distal ends of ribs 115 co-operatively engage with the sides of respective grille 114a, 114b to snap lock respective grille 114a, 114b securely in place in central aperture 112. Grille 114a is provided with a central boss 116 which is snap lockingly received in aperture 118 of complementary grille 114b when the two halves 104, 106 are assembled together. Locking flange 117 keeps boss 116 located in aperture 118 and allows relative twisting movement of the two compartments 104, 106 with respect to each other whilst preventing the two compartments 104, 106 from separating from each other. Boss 116 forms an axle around which body 106 can rotate with respect to body 104 within aperture 118 in use. Grille 114a is provided with a plurality of small indents 120 or depressions or hollows located in spaced apart locations from one another in a circular pattern outwardly around the circumference of boss 116 so as to be coaxial with boss 116.

Grille 114b is provided with a pair of cantilevered arms 122 located in diagonally opposite locations on either side of aperture 118. Arms 122 are provided with tabs 124 at their distal ends and are biased to be received and retained in the small indents 120 to retain body 104 in a selected position with respect to body 106 depending upon the relative positions required to dispense the compositions from the two compartments 104, 106 in use. The location of tabs 124 indents 120 allows one compartment 104 to be selectively rotationally positioned with respect to the other compartment 106, such as to adopt the position as shown in FIG. 7 in which compartments 104 and 106 are arranged approximately perpendicularly to each other, that is, the position of potential maximum dispensing.

Both grilles 114a, 114b are provided with a multitude of vents 130 located on either side of central boss 116 or central aperture 118 at or towards their distal ends for allowing movement of air over and through the compartments. In this embodiment each grille 114a, 114b has five arcuate air vents 130 at each end. However, any number and/or shape of vents can be present in the grilles. Also, the vents 130 can be located at any location over the area of the grilles 114a, 114b or arranged in any pattern. The closing and opening of the vents 130 adjusts the amount of composition being dispensed from the two compartments 104, 106 when the two compartments are rotationally moved with respect to each other, by altering the amount of air flow over the surface of the compositions in compartments 104, 106 and through compartments 104, 106.

In other embodiments air vents 130 of grilles 114a, 114b can be provided with removable covers or replaceable covers, in the form of strips of tape, foil or the like to selectively cover selected ones of the vents 130 of the grilles.

Optionally, grilles 114a, 114b may be provided with slide valves 134 in the form of sliding or slidable flaps or similar for covering or closing the vents 130. Flaps 134 are one example of the closures. Other examples are possible to selectively open or close vents 130. In one embodiment each cap is provided with two slide flaps 134 or similar that slide in tracks 136 provided along each edge of the caps to extend in the lengthwise extending direction of the caps. By sliding the flaps 134 lengthwise in their tracks 136 the vents 130 are selectively opened or closed to adjust the amount of air entering and flowing through the vents and hence through each body 104, 106 to dispense the respective composition therefrom.

As there are four separate slide flaps 134, one for each set of vents, the openings of each set of vents 130 is independently adjustable to dispense the contents in accordance with requirements. It is to be noted that the selective adjustors for adjusting the openings can take any form such as for example, the adjustors can be movable, slidable, rotatable, twistable, pivotable or the like.

Operation of air care dispenser 102 of this embodiment will now be described. In use, air care dispenser 102 is provided in a transport configuration as shown in FIG. 6, in which all of the four sets of vents are closed by the opposing grilles' 114a, 114b being in aligned abutting relationship with each other; this substantially prevents air movement around and through the vents 130 so that little or no composition is dispensed. When it is desired to use the dispenser 102 to dispense one or both compositions from the dispenser 102, the compartments 104, 106 are twisted with respect to each other so as to expose the vents 130. One such configuration is shown in FIG. 7 which is the fully opened position allowing air to flow through compartments 104, 106 to a maximum extent so as to dispense the two compositions at a maximum rate. This is the normal in-use position. By rotating one of compartments 104, 106 with respect to the other to a lesser or greater amount the various openings are opened to a greater or lesser amount so as to control the amount of composition being dispensed from each compartment 104, 106 in the selected in use positions.

With air care dispenser 102 in this configuration, the upper and lower edges of slides 134 are exposed so that they can be moved longitudinally along tracks 136 to adopt a selected position with respect to vents 130 so as to close some of the vents yet allow other of the vents to be opened or to fully close vents 130 or to fully open vents 130. By judiciously opening the required number of vents 130 of each set of vents to the required amount by independently moving each slide flap 134 the amount of composition in each compartment 104, 106 being dispensed can be accurately controlled thereby dispensing an overall composition having the selected composition from the dispenser. In this manner the user has control over how much of each composition can be dispensed.

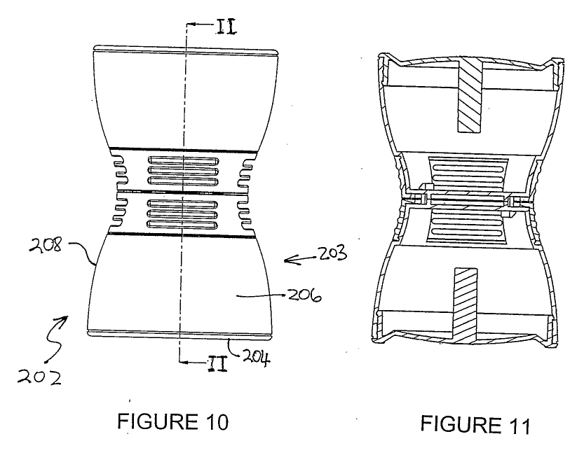

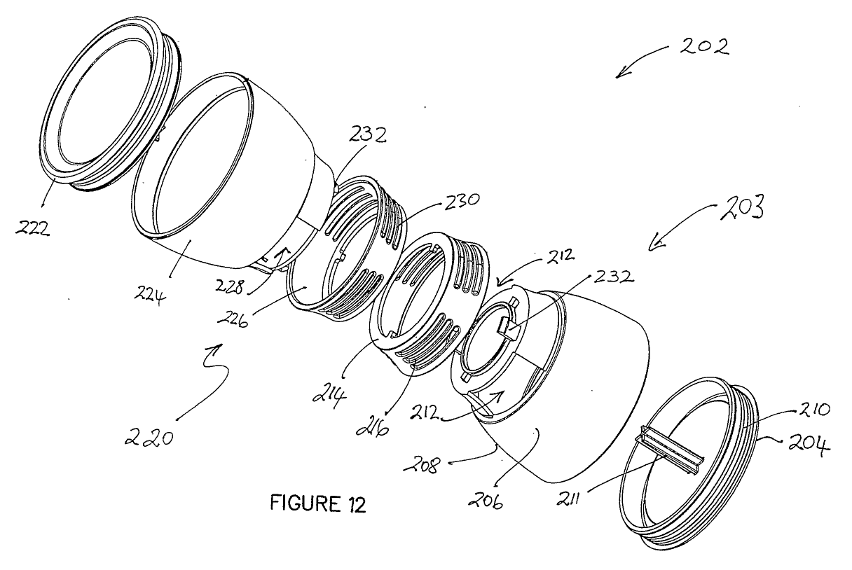

FIGS. 10 to 12 are views of a dispenser 202 according to another embodiment of the present invention. Dispenser 202 is generally in the shape of an hourglass in which one composition is located in a compartment at one end of the dispenser and another composition is located in a compartment at the other end of the dispenser.

Dispenser 202 has a first assembly 203 including a substantially circular first end cap 204 for sealing one end of an open ended substantially cylindrical first main housing 206 having a tapered wall 208. First end cap 204 is provided with a threaded portion 210 for screw threadingly being received in the distal end of first main housing 206. A first spigot 211 or similar is provided to retain the composition within the main housing 206. A pair of diagonally oppositely located first openings 212 are provided at or towards the proximal end of first main housing 206. A first collar 214 having a multiplicity of first vents 216 is received over the proximal end of first main housing 206 to cover openings 212. Vented collar 214 is free to rotate with respect to main housing 206 to selectively open and close the main housing by either partially or fully covering the openings 212. Vented collar 214 on the proximal portion of housing 206 is provided with suitable connector for connecting the first assembly to a similar assembly.

A similar construction is provided on the other side of dispenser 202 arranged as a mirror image to the first end or side of the dispenser. This other side includes a second assembly 220. The second assembly 220 which comprises second end cap 222, second main housing 224, and second vented collar 226 is rotationally connected to the first assembly 203 by a suitable connector such as retainers 232 provided on the inboard surface of the main housings allowing independent adjustment of the vents of either assembly.

In use, a first composition is located within the first compartment formed within the first main housing 206 of the first assembly 203. First end cap 204 seals the first main housing 206. A second composition is located within the compartment formed within second main housing 224 and this compartment is sealed with the second end cap 222.

To dispense the first composition, first vented collar 214 is rotated so that the first set of vents 216 move to at least be partially in register with the first openings 212 to allow air to circulate through the two pairs of aligned openings 212 and vents 216 to dispense the composition in first main housing 206 at a first rate.

Similarly, to dispense the second composition, the second vented collar 226 is rotated to align the second openings 228 of the second main housing 224 with the second vents 230 of the second vented collar 226 to allow air to circulate through and around the inside of the second main housing 224 to dispense the second composition at a second rate from the dispenser 202.

As rotation of the two vented collars 214 and 226 is independent of each other, the positions adopted by the two sets of vents 216, 230 can be independently determined so that the openings of either compartment within the respective main housings can be independently adjusted to change the rate of dispensing of the two compositions so as to dispense the two compositions independently of each other at the same rate or at different rates in accordance with requirements.

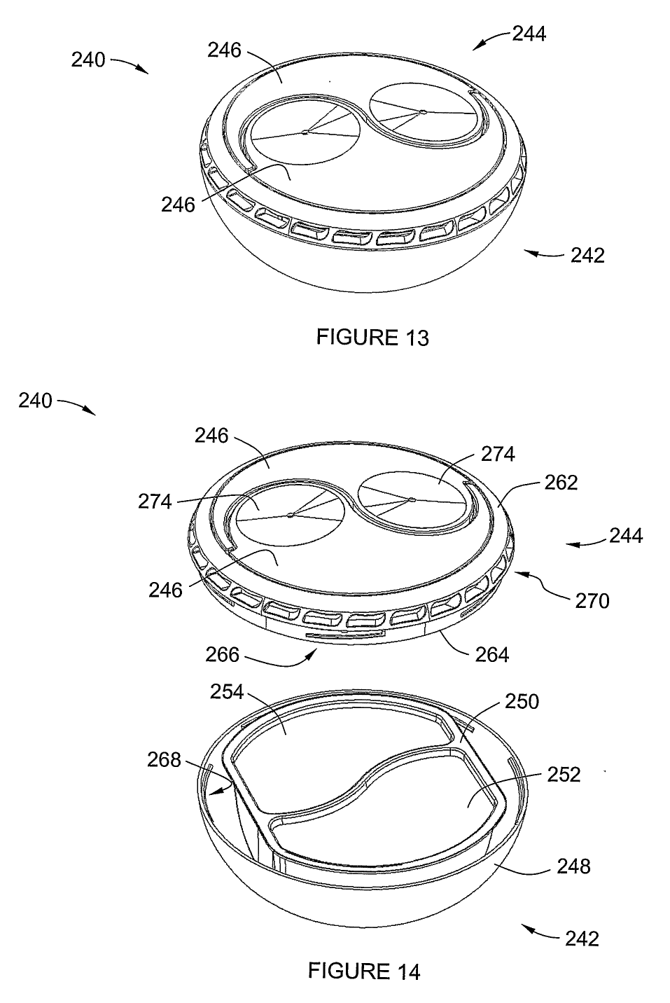

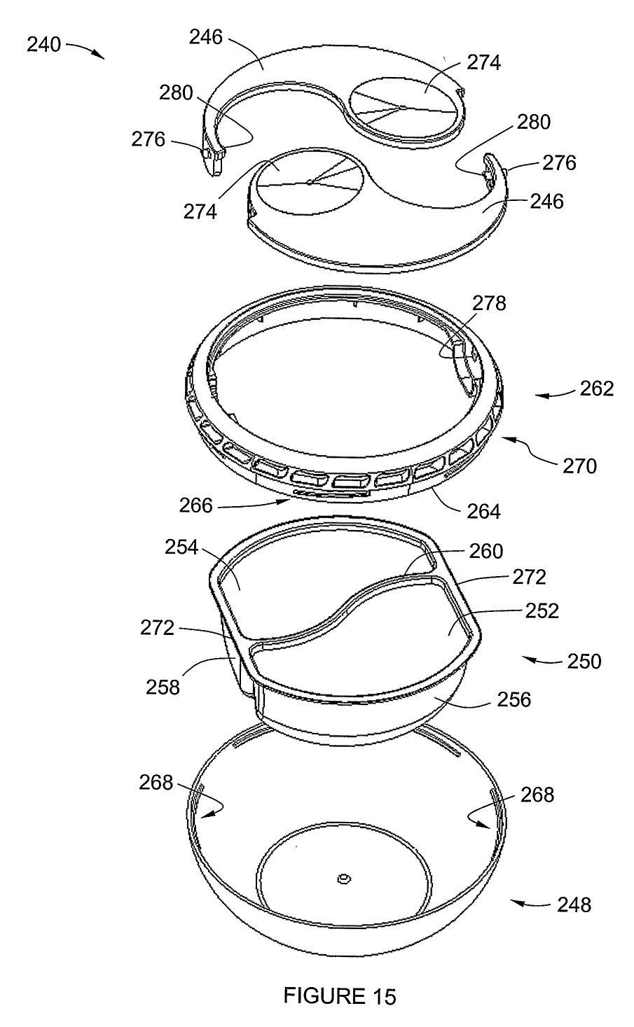

FIGS. 13 to 20 are views of an air care dispenser 240 according to another embodiment of the present invention. Dispenser 240 is similar in many respects to dispenser 2 of FIG. 1; dispenser 240 includes a base 242 and a cap 244 with two discharge mechanisms in the form of rotatably mounted, tiltable lids 246. However, unlike base 4 of dispenser 2, base 242 of dispenser 240 is not divided into two compartments by a central wall. Rather, base 242 of dispenser 240 includes a shell 248 and a container 250 of first and second air care compositions 252, 254 in respective compartments 256, 258 separated by a generally sinusoidal central wall 260; the container 250 is locatable in and removable from shell 248. This allows a user to replace container 250, such as when either or both first and second compositions 252, 254 have been exhausted, without replacing the base shell 248 or cap 244.

Cap 244 has a rim 262 provided with a downwardly projecting skirt 264, and—as mentioned above—two lids 246 rotatably mounted in rim 262. The outer periphery of skirt 264 is provided with multiple, outwardly directed thread portions 266 for engaging corresponding, inwardly directed thread portions 268 provided around the upper periphery of the inner surface of base shell 248. By this mechanism, cap 244 can be threadingly engaged with base 242. The thread portions 266, 268 of the cap 244 and base shell 248 are provided in this manner (rather than as a continuous thread) so that the thread portions 266 of cap 244 can be received between the thread portions 268 of base shell 248, and the cap 244 fully threadingly engaged with base 242 with only a partial rotation. Once secured to one another in this manner, cap 244 and base 242 cooperate to retain container 250.

Rim 262 is perforated by multiple (optional) peripheral vents 270 for allowing the contents of container 250 (i.e. compositions 252, 254) to be released; as a result, even when lids 246 are closed compositions 252, 254 will be dispensed at a minimal rate. This is made practical by the use—unlike in dispenser 2 of FIG. 1—of removable container 250. Dispenser 240 can be provided with container 250 in a sealed state, such as by being fitted with a removable plastic film covering compartments 256, 258 and adhered to the upper edge 272 of container 250. While being transported prior to sale, therefore, compositions 252, 254 will not be released and therefore wasted, notwithstanding the provision of vents 270. Refill containers 250 are provided in a similar manner, fitted with a removable plastic film covering compartments 256, 258 and adhered to upper edge 272.

Lids 246 are similar in many respects to lids 22 of dispenser 2 of FIG. 1. They are rotatably mounted (though in rim 262) to be capable of independent movement so that the extent to which each is opened can be separately controlled. This allows separate rates of release or dispensing of the two compositions 252, 254. It will be appreciated that, in use, cap 244 should be located on base 242 such that each lid 246 is located as fully over a single compartment 256, 258 (and hence a single composition 252, 254) as possible. This maximizes the extent to which the user can most fully discriminate between and independently control the release of the two compositions 252, 254.

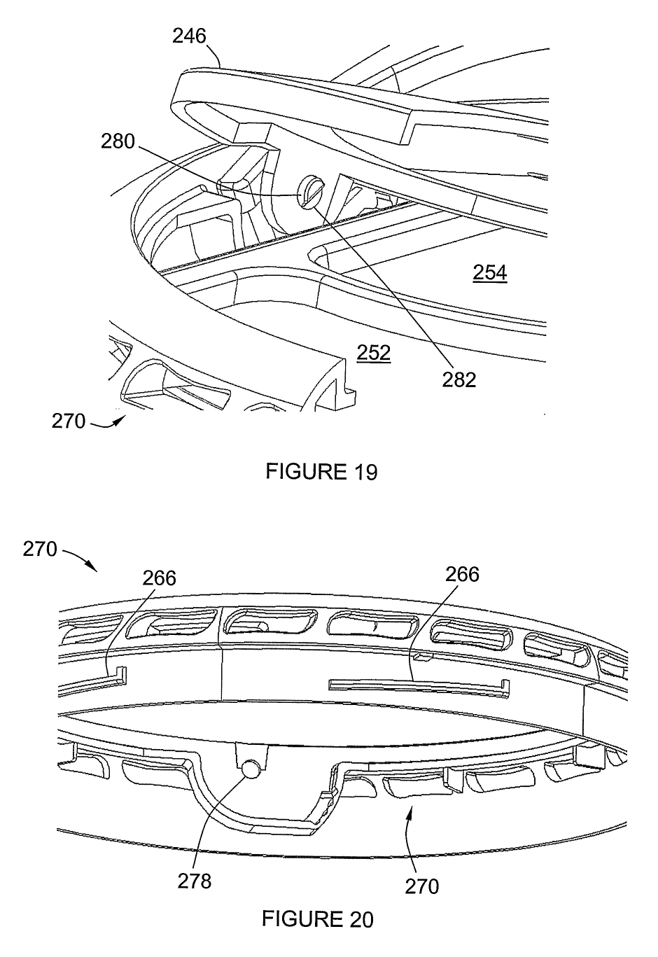

Also, like lids 22 of dispenser 2 of FIG. 1, each of lids 246 has a depression 274 at its broader end for assisting movement of the lid by guiding the user to depress the lid at the depression 274. Lids 246 also each have an outwardly directed spigot 276 at its other, narrower end. These spigots 276 are received by a pair of blind bores 278 (particularly evident in the view of FIG. 20) provided diametrically opposite each other in inner face of upper rim 262. Each of lids 246 also has an inwardly directed spigot 280 in essentially the same location as outwardly directed spigot 276, but extending inwardly and each received by an aperture 282 (see FIGS. 18 and 19) in the other lid 246 at its broader end.

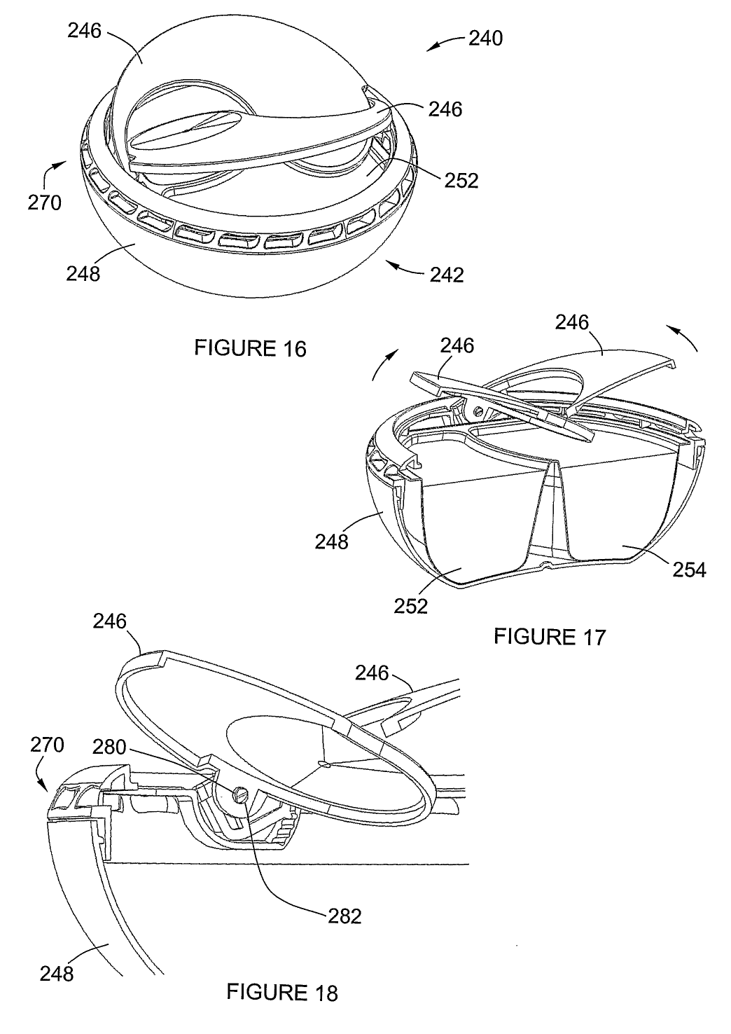

Thus, lids 246 are rotatably mounted in upper rim 246 and to each other. This is particularly evident in the views of FIGS. 16 and 17, which show the lids 246 in typical in-use configurations.

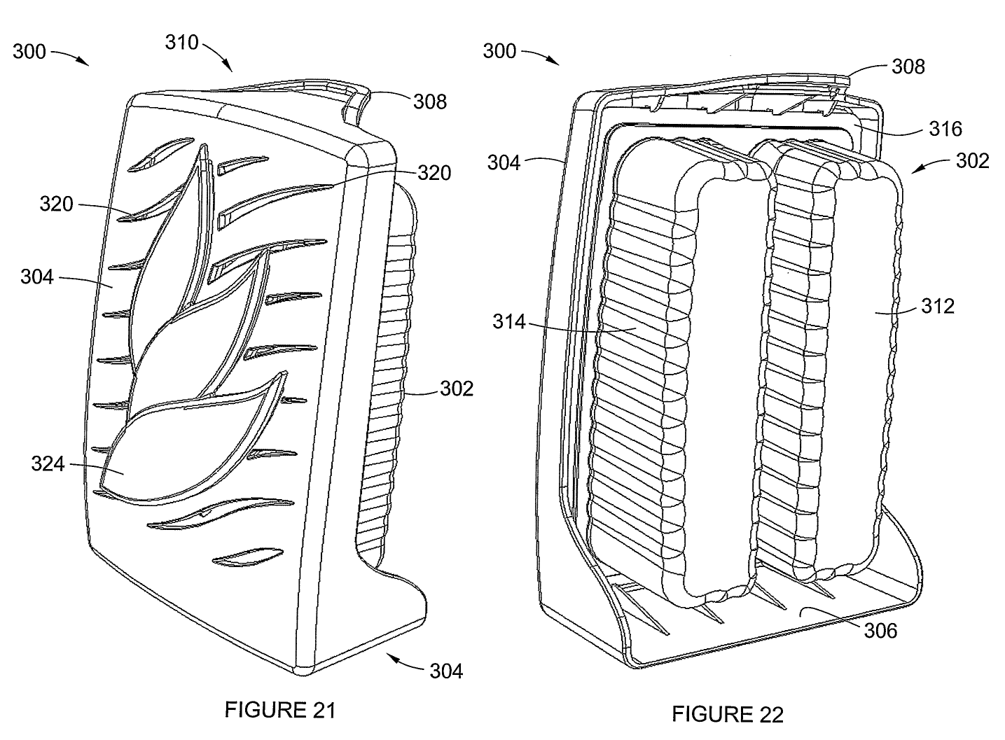

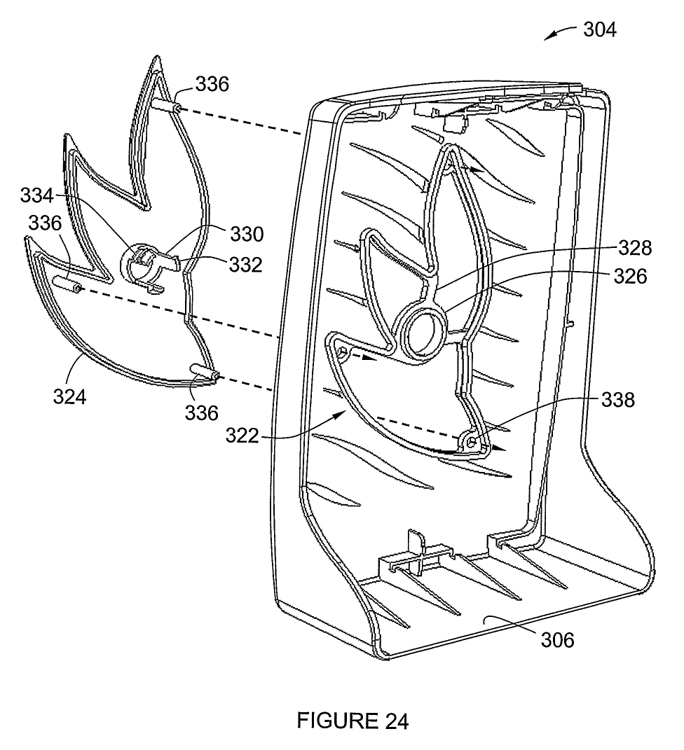

Thus, in dispenser 240 cap 244 and base shell 248 act as a housing for replaceable container 250 of first and second compositions 252, 254. In other embodiments, however, the replaceable container does not require a housing. For example, FIGS. 21 to 26 are views of an air care dispenser 300 according to another embodiment of the present invention. Dispenser 300 comprises a container 302 (for first and second air care compositions), and a front cover 304. Front cover 304 has multiple functions: it allows the user to control the dispensing of the first and second compositions, it has a base 306 so that cover 304 can act as a stand for container 302, and it has a loop 308 at its top periphery 310 so that dispenser 300 can be hung (such as from a hook).

As may be seen most clearly in the rear perspective view of FIG. 22, container 302 has two compartments 312, 314 (for, respectively, the first and second compositions), one beside the other in the view of FIG. 22. In one variation of this embodiment, the compartments 312, 314 are disposed one above the other the view of FIG. 22, and other variations are possible.

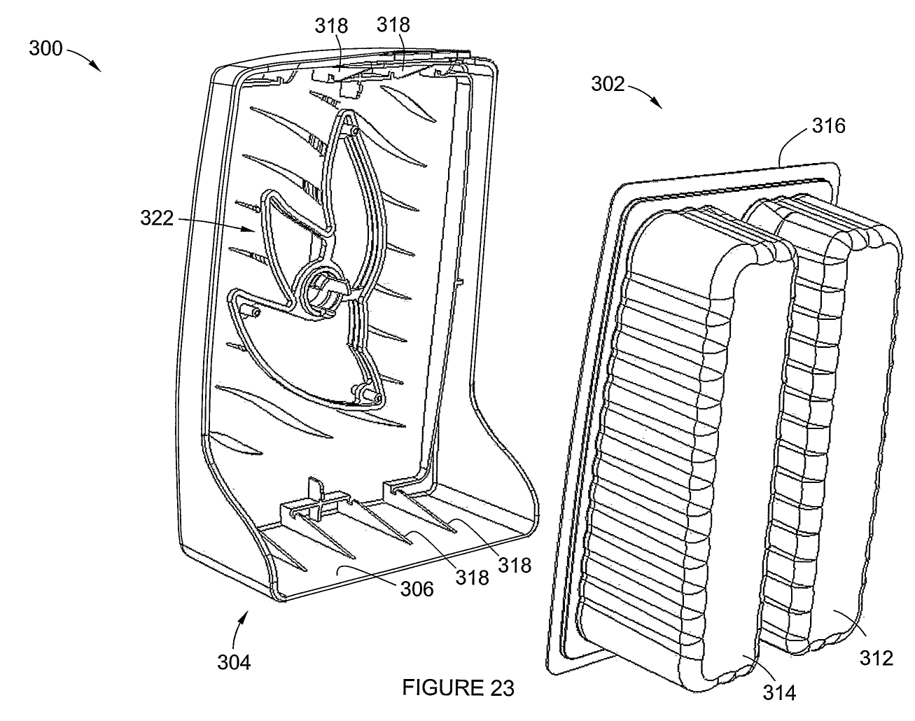

As may be seen most clearly in the rear, exploded perspective view of FIG. 23, container 302 has a peripheral lip 316; the rear of cover 304 has multiple clips 318 for engaging peripheral lip 316 to retain container 302 in place behind cover 304.

Referring to FIG. 21, front cover 304 has a series of vents in the form of slits 320 for dispensing the contents of container 302 (i.e. the two air care compositions) at a minimal rate. Front cover 304 also has a discharging mechanism in the form of closable main vent 322 and adjustable closure 324 (cf. FIG. 23). Closure 324 is located over vent 322. For decorative purposes vent 322 and closure 324 are formed to suggest a cluster of leaves. Front cover 304 has a mounting ring 326 located within vent 322, and supported by three support elements 328 extending from ring 326 to the edges of vent 322. Closure 324 has three rearwardly extending prongs 330 that define a partial cylinder locatable within ring 326. By this mechanism, closure 324 is supported over vent 322, and slidable forwardly and rearwardly to, respectively, open and close the vent 322. Each prong 330 has a tooth 332 at its rearmost end to engage ring 326 as closure 324 is drawn forward (to open vent 322), and thereby prevent prongs 330 from progressing further and disengaging ring 326. As a result, closure 324 is retained by ring 326 and hence the rest of dispenser 300.

The prongs 330 also have an outwardly directed low flange 334 at approximately half the prongs' length forward of teeth 332. Flange 334 resists but does not prevent closure 324 from being drawn forwardly, but provides a midway stop point for closure 324 between fully closed and fully open. Closure 324 can thus be conveniently located in three clearly defined stop positions: fully closed (as shown in FIG. 1), half open, and fully open. However, the resilience of the plastics material of front cover 304 (including of ring 326 and of prongs 330) allows the user to locate closure 324 at essentially any position between fully closed and fully open.

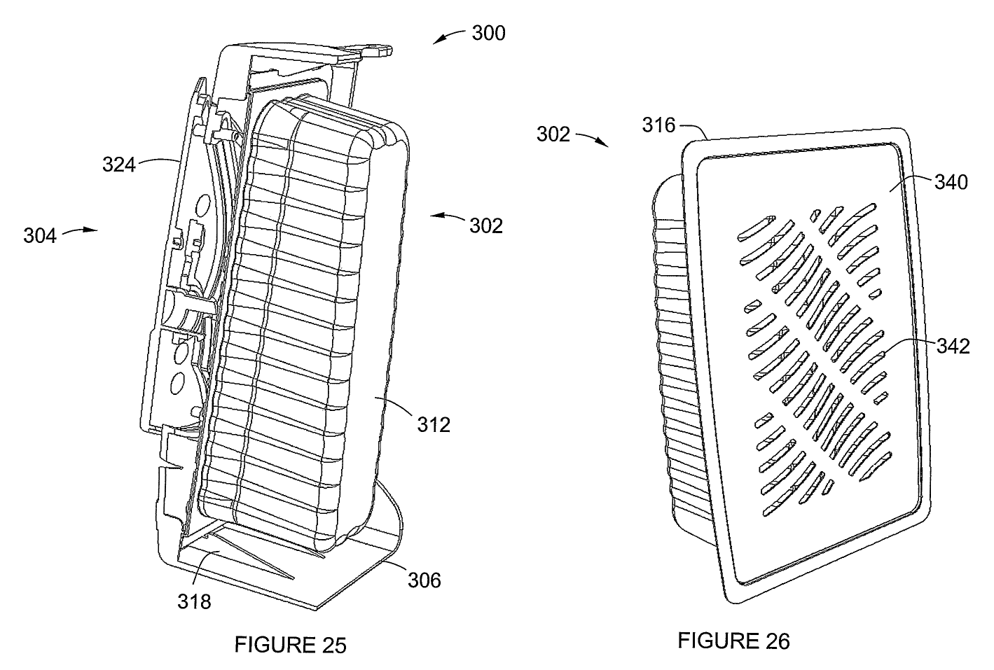

The interaction of vent 322, closure 324, ring 326 and prongs 330 are also evident from the cross-sectional view of this mechanism of FIG. 25.

It should be noted that, because vent 322 is not symmetrical, it tends to dispense one of the two compositions more than the other when closure 324 is in a partially or fully open configuration. The effect of this asymmetry will vary depending on the extent to which vent 322 is closed by closure 324, so—by varying the position of closure 324—the user can change the relative rate at which the two air care compositions are released from container 302.

Closure 324 also has three rearwardly directed guide pins 336, and front cover 304 has three corresponding apertures 338 located adjacent to vent 322 for receiving the guide pins 336. Guide pins 336 and apertures 338 cooperate to prevent closure 324 from rotating about prongs 330, so that closure 324 remains properly aligned over vent 322.

The combination of vent 322 and closure 324 thus allow the user to control the amount of the air care compositions to be dispensed, including relative to each other. A minimal amount is dispensed (through slits 320) when the closure 324 is in the fully closed configuration. A maximum amount is dispensed (through slits 320 and vent 322) when the closure 324 is in the fully opened configuration.

FIG. 26 is a front perspective view of container 302 of dispenser 300. As is apparent from this view, container 302 has a retaining lid 340 provided with vents 342 to allow the dispensing of the air care composition contents (not shown) of the container 302. Retaining lid 340 can alternatively comprise a permeable film, perforated lid, or a perforated film. As with dispenser 240 of FIG. 13, dispenser 300 can be provided with container 302 in a sealed state, such as by being fitted with a removable plastic film covering its two compartments 312, 314 and adhered to peripheral lip 316 (and hence over retaining lid 340). While being transported, therefore, the contents of container 302 will not be released, notwithstanding the provision of vents 342. Refill containers 302 are provided in a similar manner.

As has been discussed above, the first and second compositions of the above-described embodiments may be provided in a replaceable refill. Such refills (for, for example, container 250 of dispenser 240 of FIGS. 13 to 20 or container 302 of dispenser 300 of FIGS. 21 to 26) would typically be provided with a removable film seal for preserving the first and second compositions prior to use.

If the first or second compositions are provided in liquid form, a secondary film may be provided under the removable film seal. This secondary film would be sufficiently permeable that the active constituents of the first or second compositions can escape (such as through microscopic perforations), but nonetheless retain the remainder of the (liquid) first and second compositions.

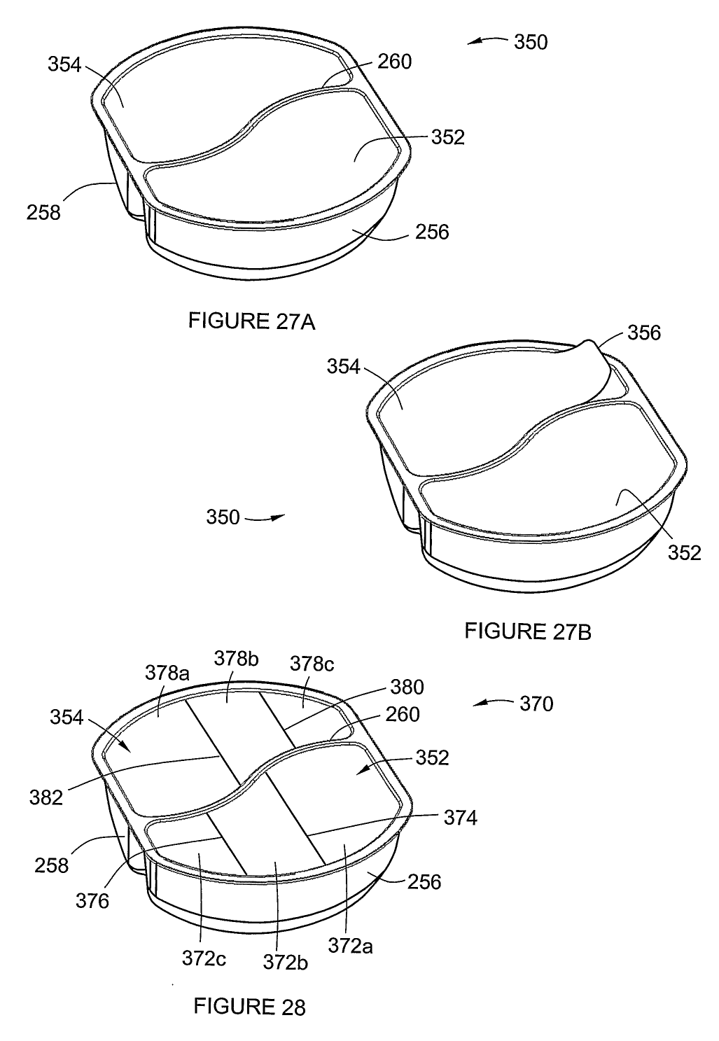

According to the present invention, however, such refills can be provided in a number of forms. FIG. 27A is a top perspective view of a refill 350 for dispenser 240 of FIG. 13. In size and general design, refill 350 is identical with container 250 of dispenser 240 (and like reference numerals have been used to indicate like features). It may optionally have a retaining lid comparable to retaining lid 340 of container 302 of dispenser 300 of FIG. 21 over its contents. However, rather than a single removable film seal, refill 350 has separate removable first and second film seals 352 and 354 for respective first and second compartments 256 and 258. This allows a user to, if desired, expose only one of the first and second compositions. For example, the first composition (in first compartment 256) may be an odour neutraliser, while the second composition (in second compartment 258) may be a fragrance. Some users may prefer to use either the fragrance or the odour neutraliser exclusively, or sequentially, and can do so by selectively removing one of the film seals 352, 354. If the other of the fragrance and odour neutraliser is required after the first is exhausted, the other film seal can then be removed. Referring to FIG. 27B, the user can also use this mechanism to control the quantity of fragrance or odour neutraliser that is released (in addition to the control provided in this regard by dispenser 240). Thus, second film seal 354 may be partially removed by lifting one end 356 of film seal 354 without entirely removing the film seal 354. In this example, a small quantity of fragrance would thereby be released. If first film seal 352 is then entirely removed, the user can obtain the full odour neutralising effect with only a limited release of fragrance. This can be then further fine-tuned by using the adjustable discharge mechanisms (i.e. lids 246) of dispenser 240.

It should be noted, in addition, that the adjustability provided by film seals 352, 354 allows refill 350 to provide independent, controlled release of its two compositions without employing dispenser 240. Thus, refill 350 may be regarded both as a refill for container 240 and also as a dispenser according to still another embodiment of the present invention.

An alternative refill for dispenser 240 of FIG. 13 is shown in top perspective view at 370 in FIG. 28. Refill 370 is similar to refill 350 of FIG. 27A, and like reference numerals have been used to indicate like features. Refill 370 differs from refill 350, however, in that each of first and second film seals 352, 354 of refill 370 is divided into three segments. Hence, first film seal 352 comprises first, second and third segments 354a, 354b, 354c, separable from each other along score lines 374, 376. Second film seal 354 comprises first, second and third segments 378a, 378b, 378c separable from each other along score lines 380, 382. The score lines 374, 376, 380, 382 both identify the segments 354a, 354b, 354c, 378a, 378b, 378c to the user and facilitate the independent removal of any of the segments.

Thus, first and second film seals 352, 354 remain integral during transport but, in use, the user can choose to remove any number of the individual segments according to the amount of respective first and second compositions the user wishes to release. The user may, for example, wish to release a large quantity of odour neutraliser and a small quantity of fragrance: this can be achieved by removing the largest segment 372a of first film seal 352 and the smallest segment 378c of second film seal 354; a large number of other permutations are possible.

This technique can be combined with the adjustability provided by lids 246 of dispenser 240 or, in common with refill 350, refill 370 can itself be used as a dispenser with adjustable discharge rates according to another embodiment of the present invention.

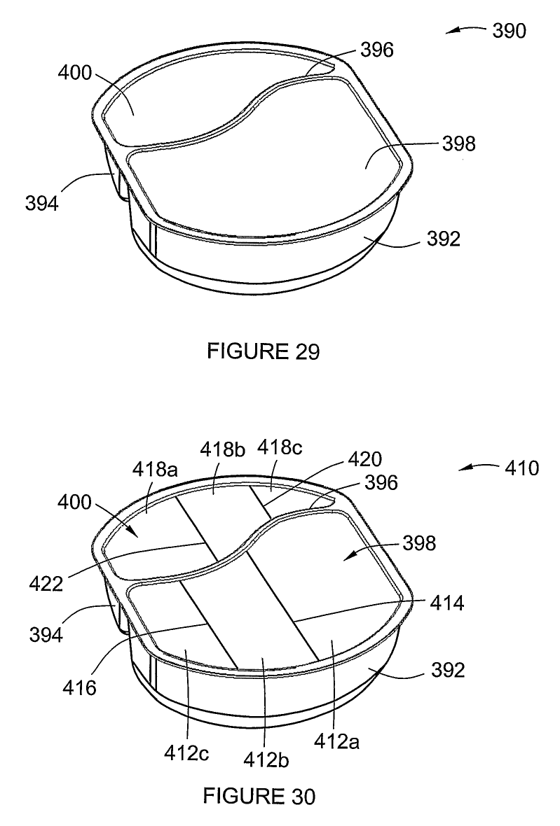

The containers and refills described thus far have identically sized first and second compartments. However, is not so in all embodiments. FIG. 29, for example, is a top perspective view of a container or refill 390 for use with a dispenser (such as dispenser 240 of FIG. 13), having first and second compartments 392, 394, where the first compartment 392 has approximately twice the volume of second compartment 394. Container or refill 390 thus can hold approximately twice the quantity of a first composition (by volume) than of a second composition. Such a container or refill would be desirable for those users who prefer, for example, a large quantity of odour neutraliser and a smaller quantity of fragrance (or vice versa). In addition, container/refill 390—in common with refill 350 of FIG. 27A—has two removable film seals, a first film seal 398 for sealing first compartment 392 prior to use and a second film seal 400 for sealing second compartment 394 prior to use. First and second film seals 398, 400 can be removed partially or fully in the manner described above by reference to FIGS. 27A and 27B.

Furthermore, the individual film seals can be provided in segmented form (cf. with refill 370 of FIG. 28). FIG. 30 is a top perspective view of a container or refill 410 (suitable also for dispenser 240 of FIG. 13), which is identical in most respects with container/refill 390 of FIG. 29. However, first film seal 398 of container/refill 410 comprises three separable segments 412a, 412b, 412c defined by score lines 414 and 416. Similarly, second film seal 400 comprises separable segments 418a, 418b, 418c, defined by score lines 420, 422. As in the case of refill 370 of FIG. 28, container/refill 410 of FIG. 30 allows the user to remove one or more segments of either or both film seals 398, 400 to control the rate of discharge of the first composition (in compartment 392) and the second composition (in compartment 394).

Containers or refills 390 and 410 are thus also further embodiments of dispensers according to the present invention.

It will be understood to persons skilled in the art of the invention that many modifications may be made without departing from the scope of the invention. For example, although the present invention has been described with particular reference to one or more embodiments of the present invention, each dispenser having at least two independently adjustable compartments, the scope of the present invention is not restricted to the described embodiments but rather the scope of the present invention is more extensive so as to encompass other embodiments and arrangements of the dispenser, refills for the dispenser, and the use of dispensers for dispensing a range of different materials in a variety of circumstances using different dispensing or delivery systems including different forms and arrangements of the dispensing mechanism and the adjustable discharging mechanism for controlling the amount of materials being dispensed.

In the claims that follow and in the preceding description of the invention, except where the context requires otherwise owing to express language or necessary implication, the word “comprise” or variations such as “comprises” or “comprising” is used in an inclusive sense, that is, to specify the presence of the stated features but not to preclude the presence or addition of further features in various embodiments of the invention.

Further, any reference herein to prior art is not intended to imply that such prior art forms or formed a part of the common general knowledge in Australia or any other country.

1. A dispenser for dispensing at least first and second compositions stored in the dispenser, said dispenser comprising an adjustable discharging mechanism for discharging said first composition at a first rate and said second composition at a second rate.

2. A dispenser as claimed in claim 1, including at least a first compartment for containing said first composition and a second compartment for containing said second composition.

3. A dispenser as claimed in claim 1, wherein said discharging mechanism is adapted to permit said first rate and said second rate to be adjusted relative to each other.

4. A dispenser as claimed in claim 1, wherein said discharging mechanism includes a first adjustable discharge mechanism for discharging said first composition at said first rate and a second adjustable discharge mechanism for discharging said second composition at said second rate, wherein said first rate and said second rate are at least partially independently adjustable.

5. A dispenser as claimed in claim 1, wherein said first composition is a first form of a composition and said second composition is a second form of said composition.

6. A dispenser as claimed in claim 1, wherein said discharging mechanism is adapted to expose said first composition and said second composition to different extents as said discharging mechanism is adjusted.

7. A dispenser as claimed in claim 6, wherein said discharging mechanism comprises a rotatable shutter that in a first position fully covers said first composition, in a second position fully covers said second composition and in a third position covers said first composition partially and said second composition to a greater or lesser extent than said discharging mechanism covers said first composition.

8. A dispenser as claimed in claim 6, wherein said discharging mechanism comprises a closure for covering a vent from which said first and second compositions are discharged, wherein said closure has a plurality of positions that permit discharging of said first and second compositions at different rates.

9. A dispenser as claimed in claim 8, including a vent that permits more venting by said first composition than by said second composition.

10. A dispenser as claimed in claim 1, wherein said dispenser has a transport configuration in which said dispenser is fully closed, and a plurality of use configurations in which said dispenser is in respective open conditions.

11. A dispenser as claimed in claim 1, wherein said first and second compositions comprise air care products or compositions.

12. A dispenser as claimed in claim 1, wherein at least one of said first and second compositions comprises an odour neutraliser or a perfume or a fragrance or a masking agent.

13. A dispenser as claimed in claim 1, wherein at least one of said first and second compositions comprises one or more essential oils or aromatherapy oils.

14. A dispenser as claimed in claim 1, wherein said discharging mechanism can be adjusted by depressing, sliding, rotating, or pivoting.

15. A dispenser as claimed in claim 1, wherein said discharging mechanism has at least three positions.

16. A dispenser as claimed in claim 1, wherein said discharging mechanism

17. A dispenser as claimed in claim 1, wherein said first and second compositions are provided in a removable container or refill.

18. A dispenser as claimed in claim 1, including a first removable seal for said first composition and a second removable seal for said second composition, wherein said first removable seal can be partially or fully removed to partially or fully expose said first composition and said second removable seal can be partially or fully removed to partially or fully expose said second composition.

19. A dispenser as claimed in claim 18, wherein said first removable seal comprises a plurality of seal segments that can be individually removed to expose said first composition by respective different amounts.

20. A method of dispensing first and second compositions from a dispenser, comprising selectively adjusting a discharging mechanism to dispense said first composition at a first rate and said second composition at a second rate.

21. A method as claimed in claim 20, wherein said discharging mechanism comprises a first discharge mechanism for controlling discharge of said first composition at said first rate and a second discharge mechanism for controlling discharge of said second composition at said second rate, said method comprising independently adjusting said first discharge mechanism and said second discharge mechanism.

22. A method as claimed in claim 20, wherein said first and second rates are relatively adjusted to select a desired effect.

23. A method as claimed in claim 20, including discharging said first composition from a first compartment of said dispenser, and discharging said second composition from a second compartment of said dispenser.

24. A method as claimed in claim 20, including independently controlling said first rate from a minimum rate to a maximum rate and said second rate from a minimum rate to a maximum rate.

25. A method as claimed in claim 20, including combining, blending or mixing said first composition and said second composition as desired so that a selected blend of said first and said second compositions are dispensed from said dispenser.

26. A refill for a dispenser for dispensing at least first and second compositions, comprising:
a container;
a first composition and a second composition; and
a removable seal for preserving said first and second compositions prior to use;
wherein said removable seal is partially removable, fully removable, or both partially and fully removable

27. A refill as claimed in claim 26, wherein said container has a first compartment for said first composition and a second compartment for said second composition.

28. A refill as claimed in claim 26, wherein said container is adapted to fit a holder having an adjustable discharging mechanism for discharging said first composition at a first rate and said second composition at a second rate.

29. A refill as claimed in claim 28, wherein said discharging mechanism permits said first rate and said second rate to be adjusted relative to each other.

30. A refill as claimed in claim 28, wherein said adjustable discharging mechanism includes a first adjustable discharge mechanism for discharging said first composition at a first rate and a second adjustable discharge mechanism for discharging said second composition at a second rate, wherein said first rate and said second rate are at least partially independently adjustable.

31. A refill as claimed in claim 26, wherein said removable seal comprises a first seal portion for said first composition and a second seal portion for said second composition, wherein said first seal portion can be partially or fully removed to partially or fully expose said first composition and said second seal portion can be partially or fully removed to partially or fully expose said second composition.

32. A dispenser as claimed in claim 31, wherein said first seal portion comprises a plurality of seal segments that can be individually removed to expose said first composition by respective different amounts.
